(19) United States
(12) Patent Application Publication
(10) Pub. No.: US 2013/0073023 A1
(43) Pub. Date: Mar. 21, 2013

(54) Stent

(71) Applicants: Rosaire Mongrain, Montreal (CA); Olivier Bertrand, Quebec City (CA); Richard Lawrence Leask, Montreal (CA)

(72) Inventors: Rosaire Mongrain, Montreal (CA); Olivier Bertrand, Quebec City (CA); Richard Lawrence Leask, Montreal (CA)

(21) Appl. No.: 13/676,704

(22) Filed: Nov. 14, 2012

Related U.S. Application Data
(63) Continuation of application No. 11/730,723, filed on Apr. 3, 2007.
(60) Provisional application No. 60/808,126, filed on May 25, 2006.

(51) Int. Cl. A61F 2/82

(52) U.S. Cl. CPC A61F 2/82
USPC 623/1.2

(57) ABSTRACT

A stent insertable in a body vessel, the body vessel defining a vessel wall. The stent includes a plurality of struts, the struts defining a substantially elongated stent passageway, the struts being configured, sized and operatively coupled to each other in a manner such that the stent is deformable between a first configuration and a second configuration. In the first configuration, the stent passageway has a first radial dimension and a first longitudinal dimension, and in the second configuration, the stent has a second radial dimension and a second longitudinal dimension, the second radial dimension being at least as large as the first radial dimension and the second longitudinal dimension being larger than the first longitudinal dimension. The stent is able to expand substantially longitudinally with the body vessel as the body vessel grows without reducing in diameter so as to reduce risks of damaging the vessel wall as the body vessel grows.

### FIELD OF THE INVENTION

The present invention relates to the field of medicine and is more particularly concerned with a stent.

### BACKGROUND OF THE INVENTION

Endovascular procedures such as stenting limit the risks associated with recurrent surgery. Typically, a stent is inserted into a blood vessel and expanded so that it reduces the extent of a stenosis present in the vessel. Adult stents have been used to treat pediatric stenoses of the pulmonary system, systemic venous system, vena cava, right ventricle outflow track, ductus arteriosus and coarctations of the aorta. Paediatric stent placement has been shown to avoid re-operation or postpone additional surgery. However, when the child or teenager grows, the blood vessel typically expands in diameter, which requires that repeated angioplasty interventions be performed to re-expand the stent. Re-dilating adult-sized stents in a paediatric patient presents a risk of plaque rupture or vessel wall trauma due to over-distension.

If an implanted stent cannot expand with the vessel, a surgical intervention is needed: The vessel section is removed along with the stent and a conduit is placed to reconstruct the vasculature. Such a surgical intervention has all the drawbacks present in any surgical procedures, including risks of complications and mortality.

In addition, surgical approaches to treating paediatric stenoses have been met with difficulty over the years, and may themselves lead to further distortion of the treated arteries. The recurrent repair often required with paediatric cardiovascular diseases can be deleterious and lethal. Vessel wound healing/scaring with repeated surgical intervention produces friable and stenotic vessels.

Another problem that is not addressed in prior art stents is that not only does the blood vessel in which the stent is inserted expand in diameter as the patient grows, but also expands in length.

Against this background, there exists a need in the industry to provide a novel stent.

An object of the present invention is therefore to provide such a stent.

### SUMMARY OF THE INVENTION

In a broad aspect, embodiments of the invention provide a stent insertable in a body vessel, the body vessel defining a vessel wall. The stent includes a plurality of struts, the struts defining a substantially elongated stent passageway, the struts being configured, sized and operatively coupled to each other in a manner such that the stent is deformable between a first configuration and a second configuration. In the first configuration, the stent passageway has a first radial dimension and a first longitudinal dimension, and in the second configuration, the stent has a second radial dimension and a second longitudinal dimension, the second radial dimension being at least as large as the first radial dimension and the second longitudinal dimension being larger than the first longitudinal dimension. The stent is able to expand substantially longitudinally with the body vessel as the body vessel grows without reducing in diameter so as to reduce risks of damaging the vessel wall as the body vessel grows.

For the purpose of this document, a strut should be interpreted as encompassing any structural element of a stent attached or otherwise connected to any other similar or different element. Typically, struts are substantially elongated, but other configurations are within the scope of the invention.

In some embodiments of the invention, the second radial dimension is larger than the first radial dimension. In these embodiments, the stent is expandable both substantially longitudinally and substantially radially so as to be able to expand both substantially longitudinally and substantially radially with the body vessel as the body vessel grows.

In some embodiments of the invention, the struts define a generally longitudinally extending backbone, the backbone being deformable between a backbone shorter configuration and a backbone longer configuration. The backbone extends longitudinally along a longer distance in the backbone longer configuration than in the backbone shorter configuration. Also, the struts define a wall supporting member for supporting the vessel wall, the wall supporting member extending substantially circumferentially from the backbone, the wall supporting member being circumferentially interrupted and defining a free end. The wall supporting member is substantially radially expandable between a supporting member retracted configuration and a supporting member expanded configuration. Upon an expansion of the wall supporting member, the free end moves relatively to the backbone.

Advantageously, the proposed stent, in some embodiments, therefore minimizes or eliminates the need to redilate or surgically remove the stent as the body vessel grows. This property is advantageous in pediatric applications, for example. In some embodiments of the invention, in use, the stent becomes embedded into the vessel wall. Then, having wall supporting members that are circumferentially interrupted and a backbone that is substantially longitudinally expandable allows the stent to expand when the body vessel grows. This is made possible by the relatively large flexibility of the circumferential members and the elongation properties of the backbone. Therefore, the stent allows a reduction in the number of interventions required to maintain the body vessel open. In some embodiments of the invention, the stent even eliminates the need for such interventions.

Furthermore, in some embodiments of the invention, the stent includes a plurality of wall supporting members that are longitudinally spaced apart from each other and which are independently expandable. In these embodiments, the body vessel may grow at different rates at different locations while allowing the stent to expand and stay embedded within the vessel wall while the body vessel grows.

Yet furthermore, the stent has a relatively small diameter in the retracted configuration. Therefore, the stent is relatively easy to insert through the relatively small body vessels of children and teenagers.

In addition, in some embodiments of the invention, the stent shows minimal or zero foreshortening when expanded. This in turn facilitates positioning the stent at a desired location, since the length of the stent in the expanded state will be substantially the same as the length in the retracted or crimped state, such that a user may more readily determine were the stent will be ultimately positioned after expansion while inserting the stent in the retracted or crimped configuration.

The stent is usable to treat stenoses such as those of the pulmonary system, systemic venous system, vena cava, right ventricle outflow track, ductus arteriosus, as well as coarctations of the aorta, among other possibilities. For example, the stenosis of the pulmonary artery is a good candidate for treatment with the proposed stent. The elastic nature of the pulmonary artery and its branches make angioplasty alone an unsuccessful treatment option due to vessel recoil, and would be a suitable example for an application of the present invention.

Other objects, advantages and features of the present invention will become more apparent upon reading of the following non-restrictive description of embodiments thereof, given by way of example only with reference to the accompanying drawings.

### BRIEF DESCRIPTION OF THE DRAWINGS

In the appended drawings:

FIG. 1, in a perspective view, illustrates a stent in accordance with an embodiment of the present invention, the stent being shown in a first configuration;

FIG. 2, in a perspective view, illustrates the stent shown in FIG. 1, the stent being shown in a second configuration;

FIG. 2A, in a perspective view, illustrates the stent shown in FIGS. 1 and 2, the stent being shown in a third configuration;

FIG. 3, in a partial perspective view, illustrates the stent shown in FIGS. 1 and 2, the stent being shown in the expanded configuration;

FIG. 4, in a partial flattened view, illustrates a backbone usable in the stent shown in FIGS. 1 to 3;

FIG. 5, in a partial flattened view, illustrates another backbone usable in the stent shown in FIGS. 1 to 3;

FIG. 6, in a flattened schematic view, illustrates a stent in accordance with an alternative embodiment of the present invention, the stent being unrolled and laid flat;

FIG. 7, in a flattened schematic view, illustrates a stent in accordance with another alternative embodiment of the present invention, the stent being unrolled and laid flat;

FIG. 8, in a partial flattened view, illustrates a hybrid of the stents shown in FIGS. 6 and 7, the stent being unrolled and laid flat;

FIG. 9, in a flattened view, illustrates a stent in accordance with yet another alternative embodiment of the present invention, the stent being unrolled and laid flat;

FIG. 10, in a flattened view, illustrates a stent in accordance with yet another alternative embodiment of the present invention, the stent being unrolled and laid flat;

FIG. 11, in a flattened view, illustrates a stent in accordance with yet another alternative embodiment of the present invention, the stent being unrolled and laid flat;

FIG. 12, in a flattened view, illustrates a stent in accordance with yet another alternative embodiment of the present invention, the stent being unrolled and laid flat;

FIG. 13, in a flattened view, illustrates wall supporting members usable in the stents shown in FIGS. 1 to 12, the wall supporting members being unrolled, laid flat and shown attached to a portion of a backbone;

FIG. 14, in a flattened view, illustrates alternative wall supporting members usable in the stents shown in FIGS. 1 to 12, the wall supporting members being unrolled, laid flat and shown attached to a portion of a backbone;

FIG. 15, in a flattened view, illustrates other alternative wall supporting members usable in the stents shown in FIGS. 1 to 12, the wall supporting members being unrolled, laid flat and shown attached to a portion of a backbone;

FIG. 16, in a flattened view, illustrates yet another alternative wall supporting member usable in the stents shown in FIGS. 1 to 12, the wall supporting member being unrolled, laid flat and shown attached to a portion of a backbone;

FIG. 17, in a flattened view, illustrates yet another alternative wall supporting member usable in the stents shown in FIGS. 1 to 12, the wall supporting member being unrolled, laid flat and shown attached to a portion of a backbone;

FIG. 18, in a flattened view, illustrates yet another alternative wall supporting member usable in the stents shown in FIGS. 1 to 12, the wall supporting member being unrolled, laid flat and shown attached to a portion of a backbone;

FIG. 19, in a flattened view, illustrates yet another alternative wall supporting member usable in the stents shown in FIGS. 1 to 12, the wall supporting member being unrolled, laid flat and shown attached to a portion of a backbone;

FIG. 20, in a flattened view, illustrates yet another alternative wall supporting member usable in the stents shown in FIGS. 1 to 12, the wall supporting member being unrolled, laid flat and shown attached to a portion of a backbone;

FIG. 21, in a flattened view, illustrates yet another alternative wall supporting member usable in the stents shown in FIGS. 1 to 12, the wall supporting member being unrolled, laid flat and shown attached to a portion of a backbone;

FIG. 22, in a flattened view, illustrates yet another alternative wall supporting member usable in the stents shown in FIGS. 1 to 12, the wall supporting member being unrolled, laid flat and shown attached to a portion of a backbone;

FIG. 23, in a flattened view, illustrates yet another alternative wall supporting member usable in the stents shown in FIGS. 1 to 12, the wall supporting member being unrolled, laid flat and shown attached to a portion of a backbone;

FIG. 24, in a flattened view, illustrates yet another alternative wall supporting member usable in the stents shown in FIGS. 1 to 12, the wall supporting member being unrolled, laid flat and shown attached to a portion of a backbone;

FIG. 25, in a flattened view, illustrates yet another alternative wall supporting member usable in the stents shown in FIGS. 1 to 12, the wall supporting member being unrolled, laid flat and shown attached to a portion of a backbone;

FIG. 26, in a flattened view, illustrates yet other alternative wall supporting members usable in the stents shown in FIGS. 1 to 12, the wall supporting members being unrolled, laid flat and shown attached to a portion of a backbone;

FIG. 27, in a perspective view, illustrate a wall supporting member usable in the stents shown in FIGS. 1 to 12, the wall supporting member having a substantially transversal substantially square cross-section and shown unrolled and laid flat;

FIG. 28, in a front elevation view, illustrates the wall supporting member shown in FIG. 27;

FIG. 29, in a top plan view, illustrates the wall supporting member shown in FIGS. 27 and 28;

FIG. 30, in a perspective view, illustrate a wall supporting member usable in the stents shown in FIGS. 1 to 12, the wall supporting member having a substantially transversal substantially ellipsoidal cross-section;

FIG. 31, in a front elevation view, illustrates the wall supporting member shown in FIG. 30;

FIG. 32, in a top plan view, illustrates the wall supporting member shown in FIGS. 30 and 31;

FIG. 33, in a perspective view, illustrate a wall supporting member usable in the stents shown in FIGS. 1 to 12, the wall supporting member having a substantially transversal substantially circular cross-section;

FIG. 34, in a front elevation view, illustrates the wall supporting member shown in FIG. 33;

FIG. 35, in a top plan view, illustrates the wall supporting member shown in FIGS. 33 and 34;

FIG. 36, in a perspective view, illustrate a wall supporting member usable in the stents shown in FIGS. 1 to 12, the wall supporting member having a substantially transversal substantially ovoid cross-section;

FIG. 37, in a front elevation view, illustrates the wall supporting member shown in FIG. 36;

FIG. 38, in a top plan view, illustrates the wall supporting member shown in FIGS. 36 and 37;

FIG. 39, in a perspective view, illustrate a stent in accordance with yet another embodiment of the present invention;

FIG. 40, in a partial schematic view, illustrates a stent in accordance with yet another embodiment of the present invention;

FIG. 41, in a flattened view, illustrates a stent in accordance with yet another embodiment of the present invention, the stent being shown unrolled and laid flat; and

FIG. 42, in a flattened view, illustrates a stent in accordance with yet another embodiment of the present invention, the stent being shown unrolled and laid flat.

### DETAILED DESCRIPTION

With specific reference now to the drawings in detail, it is stressed that the particulars shown are by way of example and for purposes of illustrative discussion of certain embodiments of the present invention only. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

FIGS. 1, 2, 2A and 3 illustrate a stent 10 in accordance with an embodiment of the present invention. The stent 10 is insertable in a body vessel (not shown), the body vessel defining a vessel wall (not shown). The stent includes a plurality of struts 12, the struts 12 defining a substantially elongated stent passageway 14. The stent 10 is substantially elongated and defines a stent longitudinal axis 11.

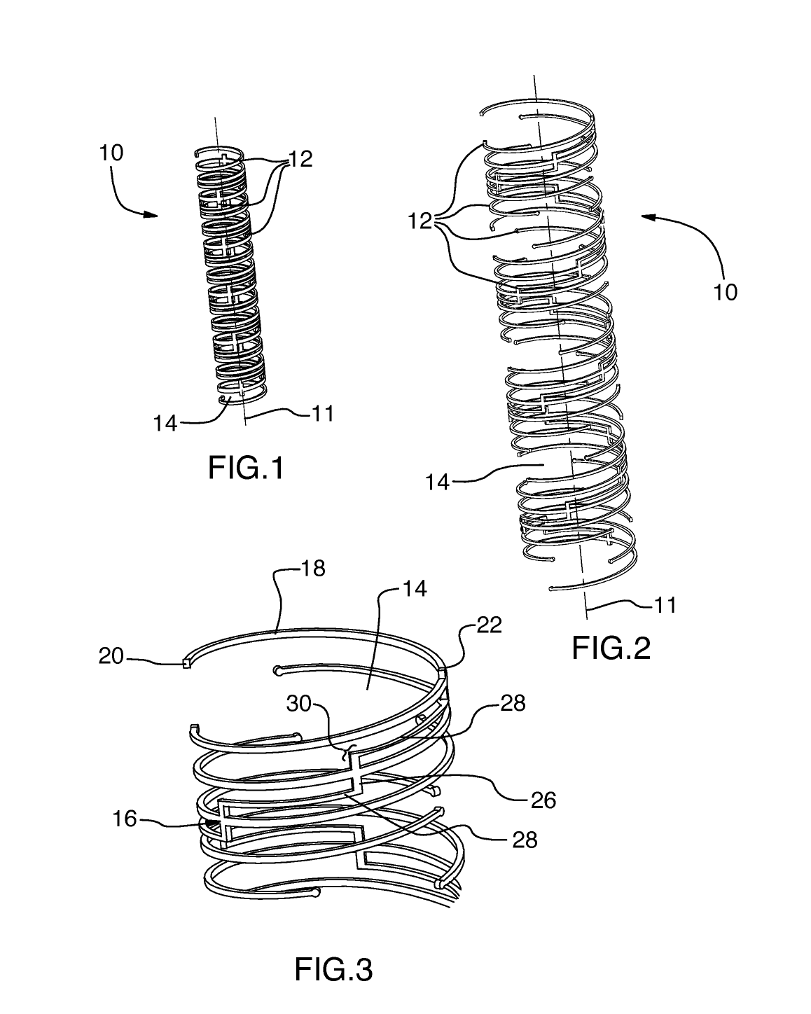

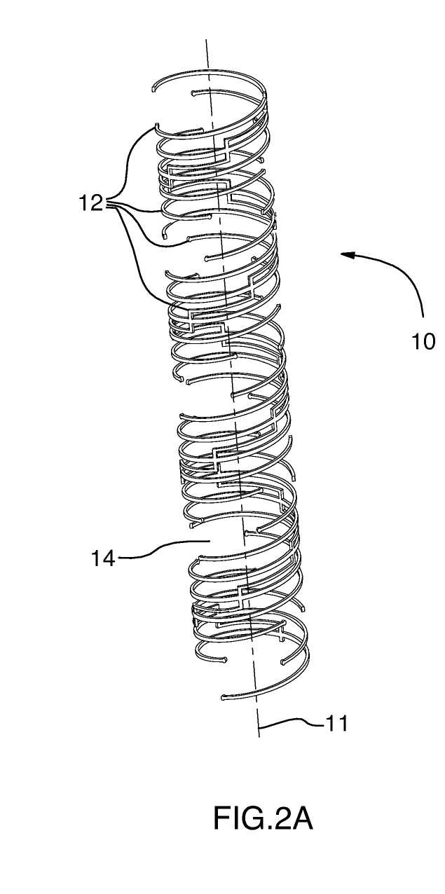

As will be further described herein below, the struts 12 are configured, sized and operatively coupled to each other in a manner such that the stent 12 is deformable between a first configuration and a second configuration. In the first configuration, the stent passageway has a first radial dimension and a first longitudinal dimension and in the second configuration, the stent passageway has a second radial dimension and a second longitudinal dimension. The second radial dimension is at least as large as the first radial dimension and the second longitudinal dimension is larger than the first longitudinal dimension. For example, once the stent 10 has been implanted within a body vessel and expanded to a first configuration, it may then be re-expanded, at least longitudinally and optionally radially as well, to a second configuration, where the length of the stent 10 in the second configuration is greater than in the first configuration. This change in configuration is illustrated by comparing the configurations of the stent 10 seen in FIGS. 2 and 2A (for a longitudinal expansion) and the stent 10 as seen in FIGS. 1 and 2 (for longitudinal and radial expansion).

Therefore, the stent is able to expand substantially longitudinally with the body vessel as the body vessel grows without reducing in diameter so as to reduce risks of damaging the vessel wall as the body vessel grows. In some embodiments of the invention, as shown in FIGS. 1 and 2, the second radial dimension is larger than the first radial dimension. In these embodiments, the stent is therefore expandable both substantially longitudinally and substantially radially so as to be able to expand both substantially longitudinally and substantially radially with the body vessel as the body vessel grows. In other words, having sufficient longitudinal, and optionally radial, expandability, allows stent 10 to grow substantially with the body vessel.

For example, it has been found that a stent 10 in which the second longitudinal dimension is at least 2.5 times larger than the first longitudinal dimension, i.e. which is operable to expand longitudinally by a factor of 2.5, and in which the second radial dimension is at least 2.5 times larger than the first radial dimension, i.e. which is operable to expand radially by a factor of 2.5, is advantageous as it allows to follow a typical growth curve of a blood vessel over a relatively large age interval in a growing patient. Also, it has been found advantageous, in some embodiments of the invention, to have a stent in which the longitudinal expandability is substantially similar to the radial expandability, as once again this allows for following approximatively the growth of body vessels in children and teenagers.

It has been hypothesized, without any intent to limit the scope of the invention, that such a stent 10 may become embedded in the vessel wall after implantation. In this case, the body vessel gradually expands the stent as it grows, which minimizes risks of injuring the body vessel and minimizes the need to manually expand the stent to compensate for vessel growth.

In some embodiments of the invention, for example in the stent 10, the struts 12 define a circumferentially interrupted structure. For example, as better illustrated in FIG. 3, the struts 12 define a generally longitudinally extending backbone 16. In the embodiments of FIGS. 1 to 3, the backbone 16 includes substantially longitudinally extending sections 26 and linking segments 28 extending therebetween. For example, and non-limitingly, the linking segments 28 are substantially arc segment shaped and the substantially longitudinally extending sections 26 are substantially rectilinear. Also, the backbone 16 includes substantially deformable sections 30 between substantially adjacent substantially longitudinally extending sections 26 and linking segments 28. In some embodiments of the invention, the substantially deformable sections 30 include a hinge formed at the intersection of the substantially longitudinally extending sections 26 and linking segments 28.

In some embodiments, the backbone 16 is deformable between a backbone shorter configuration and a backbone longer configuration. The backbone 16 extends longitudinally along a longer distance in the backbone longer configuration than in the backbone shorter configuration.

The struts 12 also define at least one, and typically a plurality of, wall supporting members 18 for supporting the vessel wall. The wall supporting members 18 extend substantially circumferentially from the backbone 16. The wall supporting members 18 are each circumferentially interrupted and each defines a respective free end 20. Furthermore, each wall supporting member 18 defines a respective fixed end 22 substantially opposed to its respective free end 20. Each wall supporting member 18 extends from the backbone 16 substantially adjacent the fixed end 22.

The wall supporting members 18 are substantially radially expandable between a supporting member retracted configuration (shown in FIG. 1) and a supporting member expanded configuration (shown in FIG. 2). Upon an expansion of the wall supporting members 18, the free ends 20 move relative to the backbone 16. In some embodiments of the invention, each of the wall supporting members 18 consists of a single strut 12 from the plurality of struts 12.

The stent 10 includes any suitable number of wall supporting members 18. In some embodiments of the invention, as better shown in FIG. 3, the wall supporting members 18 are substantially arc segment-shaped. More specifically, in the supporting member expanded configuration, the wall supporting members 18 take the form of helicoidal arc segments defining a helix axis that is substantially parallel to the stent longitudinal axis. In other words, if the wall supporting members 18 were circumferentially continuous, they would form a helical structure. However, in alternative embodiments of the invention, only a portion of each wall supporting member 18 is arc segment-shaped. In some embodiments of the invention, as seen in FIG. 3, the wall supporting members extend pairwise in substantially opposite directions from the backbone 16. For example, two wall supporting members 18 together form a substantially helicoidal arc segment having a substantially constant pitch.

The substantially longitudinally extending sections 26 are typically substantially circumferentially and substantially longitudinally spaced apart from each other. However, in alternative embodiments of the invention, the substantially longitudinally extending sections 26 are either only substantially longitudinally or only substantially circumferentially spaced apart from each other.

For example, and non-limitingly, after the stent 10 has been implanted in a patient, the substantially longitudinally extending sections 26 are circumferentially spaced apart from each other by an angle of from about 45 degrees to about 360 degrees, for example, and non-limitingly, about 45, about 60, about 90, about 120 or about 180 degrees.

The above-described structure for the backbone 16 has been found to provide good support to the vessel wall while providing a stent 10 that is relatively flexible in bending so as to conform to tortuous body vessels and to facilitate insertion of the stent 10 through body vessels to its implantation site.

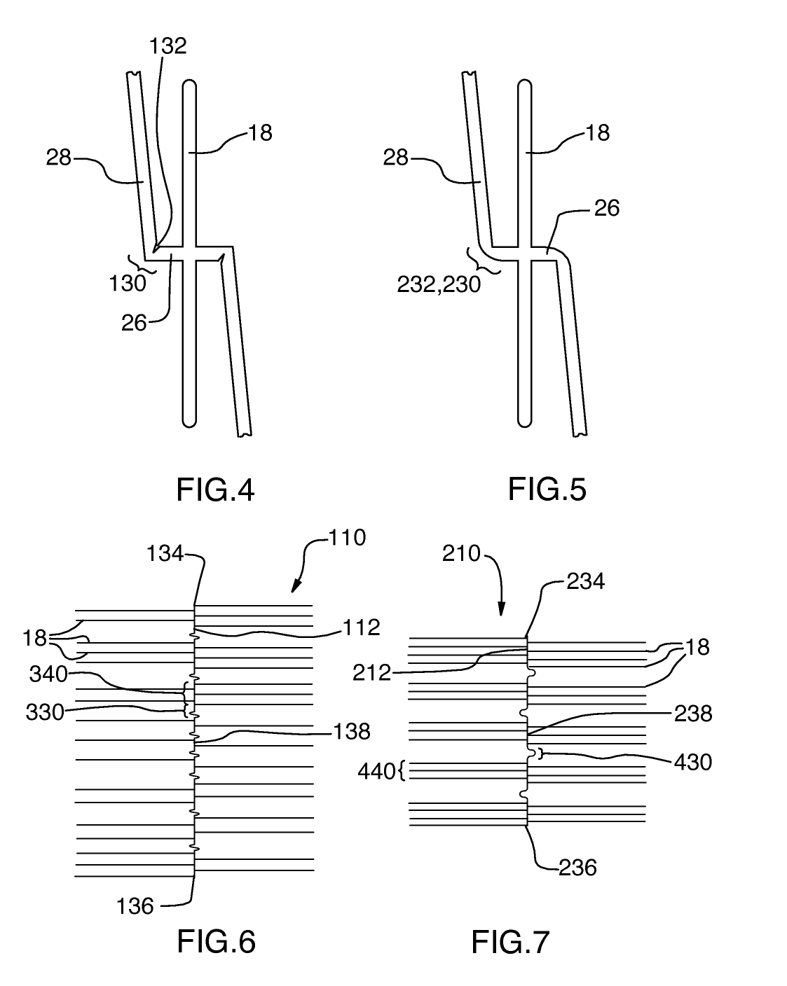

As seen in FIG. 4, in some embodiments of the invention, an alternative deformable region 130 includes a notch 132 extending into the backbone 16 at a location at which the substantially longitudinally extending sections 26 and linking segments 28 intersect. The notch 132 enhances the flexibility of the backbone 16 so as to facilitate an elongation thereof. In yet other embodiments of the invention, as seen in FIG. 5, a deformable region 230 includes a thinned out section 232 at a location at which the substantially longitudinally extending sections 26 and linking segments 28 intersect.

In some embodiments of the invention, as seen in FIGS. 6 and 7 for example, alternative stents 110 and 210 include respectively substantially rectilinear backbones 112 and 212. The backbones 112 and 212 each define a backbone first end 134, 234, a longitudinally opposed backbone second end 136, 236 and a backbone midpoint 138, 238 located substantially midway between the backbone first and second ends 132, 232 and 134, 234. In these embodiments, wall supporting members 18 extending in opposite directions are substantially longitudinally spaced apart from each other.

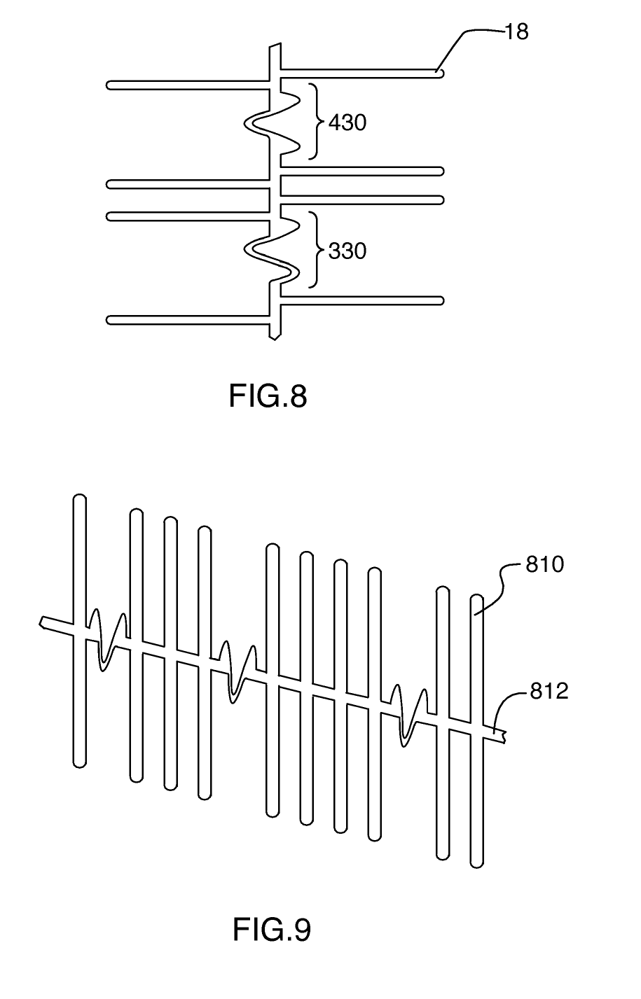

The backbones 112 and 212 include respectively substantially deformable regions 330 and 430 extending between substantially longitudinally spaced apart substantially longitudinally extending sections 340 and 440. The substantially deformable regions 330 are substantially S-shaped, while the substantially deformable regions 430 are substantially U-shaped. However, in alternative embodiments of the invention, substantially deformable regions of an alternative stent take any other suitable shapes. As seen in FIG. 8, which illustrates both types of substantially deformable regions, the substantially deformable regions 330 and 430 typically include a thinned out section having a cross-sectional area that is substantially smaller than a cross-sectional area of the reminder of the backbones 112 and 212 to facilitate the deformation thereof. Also, in some embodiments of the invention, the substantially deformable regions 330 and 430 attach to sections of the backbone having a cross-sectional area that is substantially larger than a cross-sectional area of the reminder of the backbones 112 and 212 to reduce the risk of breakage at this site of attachment.

In some embodiments of the invention, for example in the stent 10, the wall supporting members 18 are substantially similar to each other and are substantially uniformly longitudinally spaced apart from each other. In these embodiments of the invention, the stent 10 has a rigidity in a substantially radial orientation that is longitudinally substantially uniform.

In other embodiments of the invention, the wall supporting members 18 form a substantially radially most rigid region and a substantially radially least rigid region along the stent 10. The radially least and most rigid regions are substantially longitudinally spaced apart from each other. These variations allow for adapting the rigidity of the stent to the configuration of its implantation site.

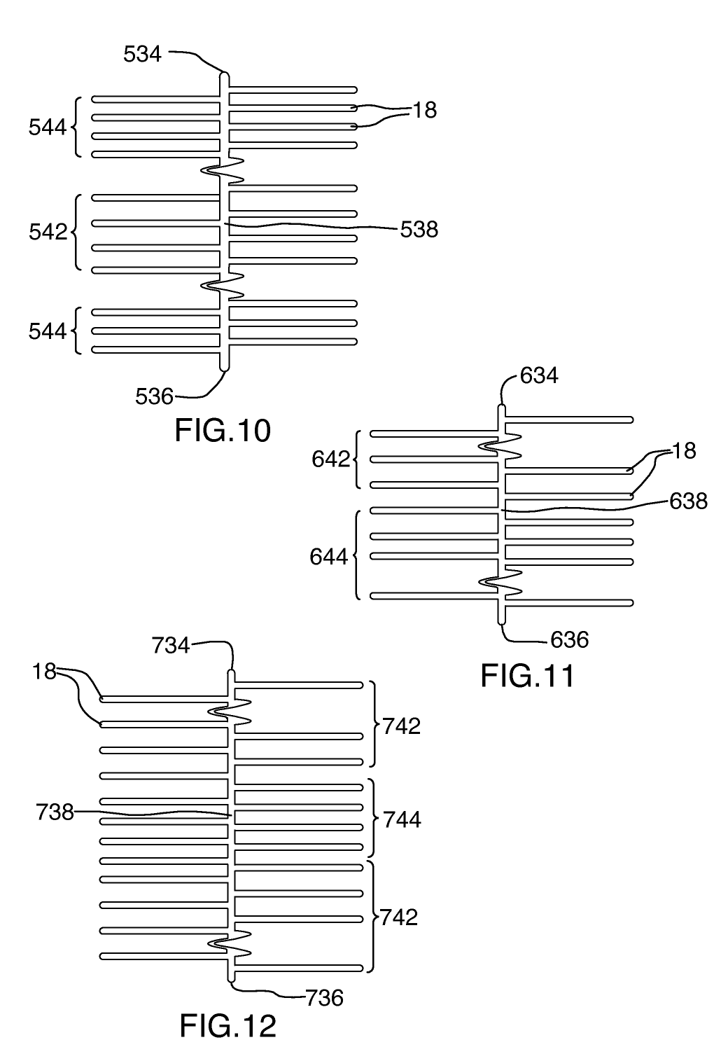

For example, as seen in FIGS. 10, 11 and 12, the wall supporting members 18 are longitudinally spaced apart by a larger distance within the radially least rigid region 542, 642 and 742 than within the radially most rigid region 544, 644 and 744. In the stent illustrated in FIG. 10, the radially least rigid region 542 is located substantially adjacent the backbone midpoint 538 and two radially most rigid regions 544 are located substantially adjacent the backbone first and second ends 534 and 536. This may be achieved by designing the stent 10 such that a substantially longitudinal distance between adjacent wall supporting members 18 increases from locations substantially adjacent the backbone first and second ends 534 and 536 towards a location substantially adjacent the backbone midpoint 538.

In the stent illustrated in FIG. 12, the locations of the radially most and least rigid regions 644 and 642 are reversed as compared to the stent of FIG. 10. This is achieved by designing the stent such that a substantially longitudinal distance between adjacent wall supporting members 18 decreases from locations substantially adjacent the backbone first and second ends 734 and 736 towards a location substantially adjacent the backbone midpoint 738. In the stent illustrated in FIG. 11, the radially least and most rigid regions 642 and 644 are respectively substantially adjacent the backbone first and second ends 634 and 636.

In other embodiments of the invention, a differential in radial rigidity within a stent according to the invention is achieved with wall supporting members 18 that extend over a larger angle within a radially most rigid region, for example in the radially most rigid region 844 shown in FIG. 41, than within a radially least rigid region, for example in the radially least rigid region 842 shown in FIG. 41. In yet other embodiments of the invention, the wall supporting members 18 each have a respective cross-sectional area in a plane extending substantially perpendicularly to a circumference of the stent 10 at an angular position relative to the backbone 16. In these embodiments of the invention, the wall supporting members 18 form a substantially radially most rigid region and a substantially radially least rigid region by having wall supporting members of a larger cross-sectional area within the radially most rigid region, for example in the radially most rigid region 944 shown in FIG. 42, than within the radially least rigid region, for example in the radially least rigid region 942 shown in FIG. 42.

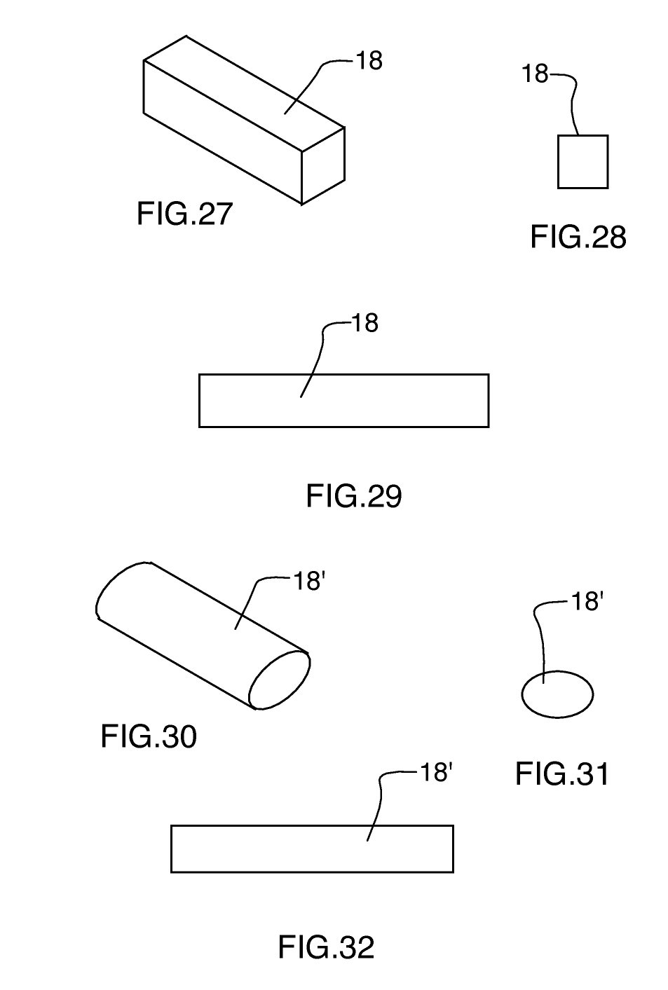

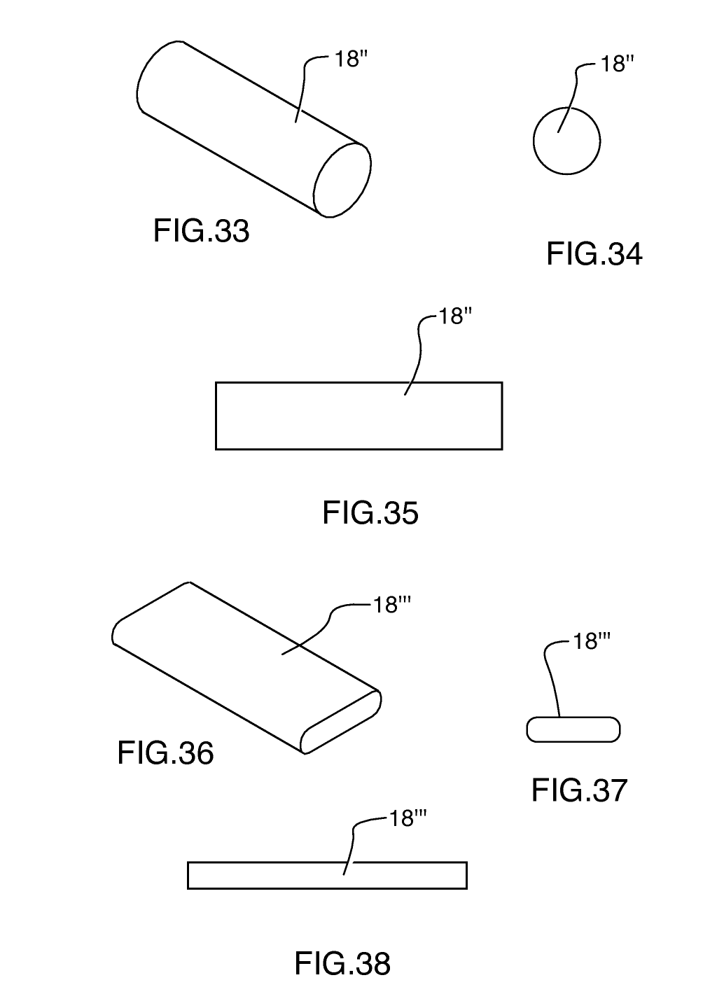

The wall supporting members 18 have any suitable cross-sectional configuration. For example, wall supporting members 18, 18′, 18″ and 18′″ may have a cross-section selected from: a substantially rectangular or square cross-section (as seen in FIGS. 27 to 29), a substantially ellipsoidal cross-section (as seen in FIGS. 30 to 32), a substantially circular cross-section (as seen in FIGS. 33 to 35) and a substantially ovoid cross-section (as seen in FIGS. 36 to 38), among others.

In some embodiments of the invention, as seen for example in FIGS. 1 to 3, the wall supporting members 18 have a cross-sectional configuration that is substantially uniform between the fixed and free ends 22 and 20. In other embodiments of the invention, the wall supporting members 18 have a cross-sectional configuration that varies between the fixed and free ends 22 and 20.

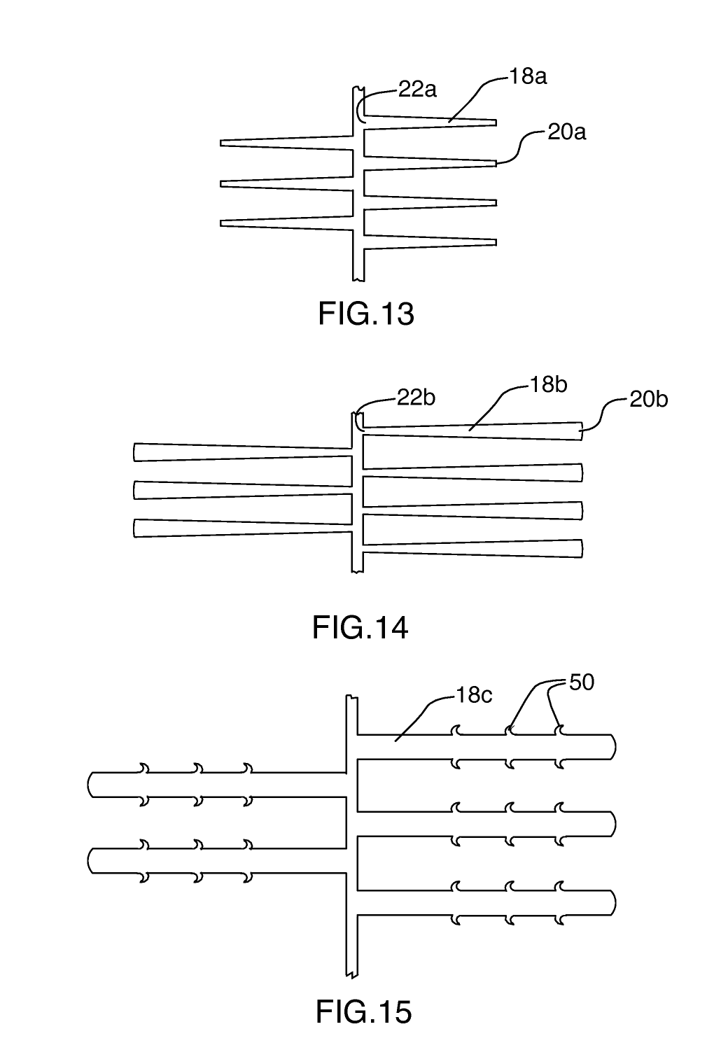

For example, as seen in FIG. 13, the wall supporting members 18a of a stent are tapered in a direction leading from their fixed end 22a towards their free end 20a. In this example, the wall supporting members 18a are substantially less rigid close to their free end 20a than close to their fixed end 22a to reduce risks of injuries that may be caused to the body vessel by the free end 20a.

Similar results are obtainable by selecting wall supporting members in which each of the wall supporting members has a material composition that varies between their fixed and free ends in a manner such that the rigidity of the wall supporting member varies to achieve this result.

In another example, as seen in FIG. 14, the wall supporting members 18b of a stent are tapered in a direction leading from their free end 20b towards their fixed end 22b. In this example, in some embodiments, the stent has a substantially circumferentially uniform rigidity in a substantially radial direction. Indeed, a reduction in radial rigidity caused by an increasing distance from the fixed end 22b is at least in part compensated by an enlargement in cross-sectional area of the wall supporting member 18b as the distance from fixed end 22b increases.

In some embodiments of the invention, as seen in FIG. 15, the wall supporting members 18c of a stent are designed to allow an expansion thereof towards their respective expanded configurations while preventing a contraction thereof towards their respective retracted configurations. For example, this is achieved by having hooks 50 that extend from the wall supporting members 18c. The hooks 50 are positioned between adjacent wall supporting members 18c and oriented to allow an expansion of the wall supporting members 18c but to prevent a contraction of the wall supporting members 18c. Indeed, the hooks 50 of adjacent wall supporting members 18c cooperate with each other to allow movement of these wall supporting members 18c relatively to each other in a single direction.

Returning to FIG. 1, the wall supporting members 18 are expandable independently from each other. Therefore, the stent 10 defines sections that are expandable in a substantially radial direction independently from each other. For example, each section includes a pair of wall supporting members 18 that extend in substantially opposite directions. In alternative embodiments of the invention, each section includes more than one pair of wall supporting members.

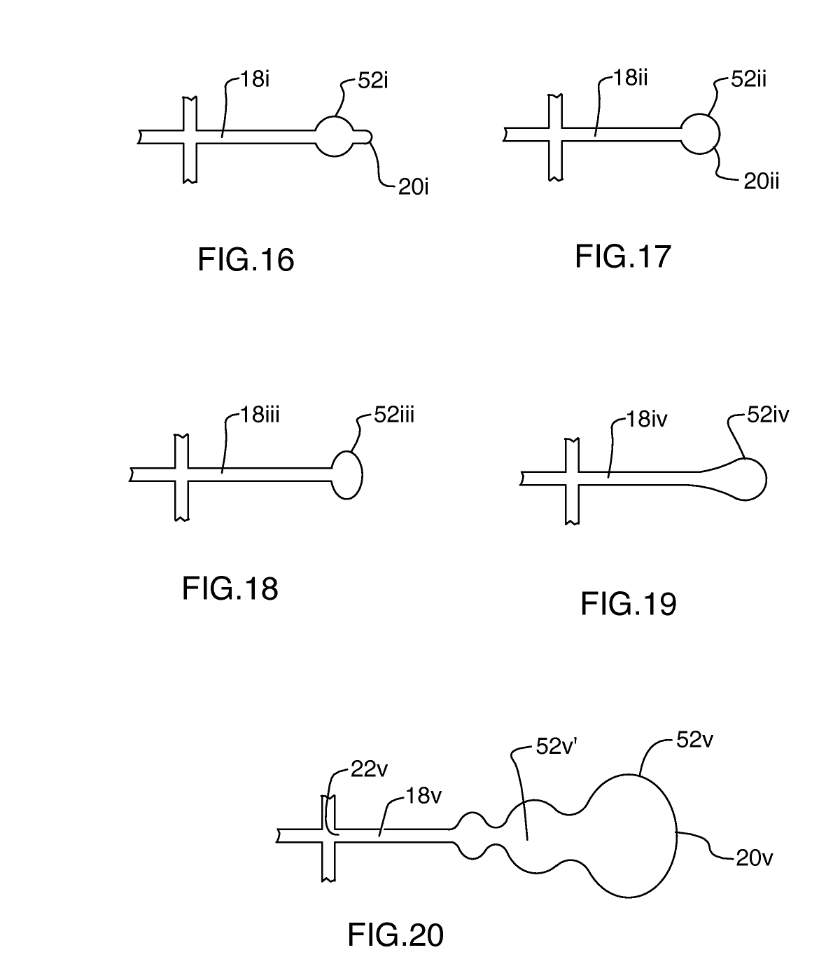

In some embodiments of the invention, as seen in FIG. 16, the wall supporting member 18i defines an anchoring section 52i for anchoring the wall supporting member 18i to the vessel wall. For example, the anchoring section 52i is substantially spaced apart from the free end 20i of the wall supporting member 18i. In alternative embodiments of the invention, as seen in FIG. 17, the anchoring section 52ii is located substantially adjacent the free end 20ii of an alternative wall supporting member 18ii.

The anchoring sections 52i and 52ii enhance the binding of the wall supporting members 18i and 18ii with the vessel wall and therefore minimize the risks that the wall supporting members 18i and 18ii become detached from the vessel wall, which could injure the vessel wall and increase the risk of formation of a thrombus within the body vessel. In addition, the anchoring sections 52i and 52ii facilitate radial expansion during body vessel growth.

In some embodiments of the invention, as seen in FIG. 20, wall supporting member 18v defines at least two anchoring sections 52v and 52v′. The at least two anchoring sections 52v and 52v′ are substantially circumferentially (relatively to the stent) spaced apart from each other. In other words, the anchoring sections 52v and 52v′ are spaced apart from each other between the fixed and free ends 22v and 20v of the wall supporting member 18v.

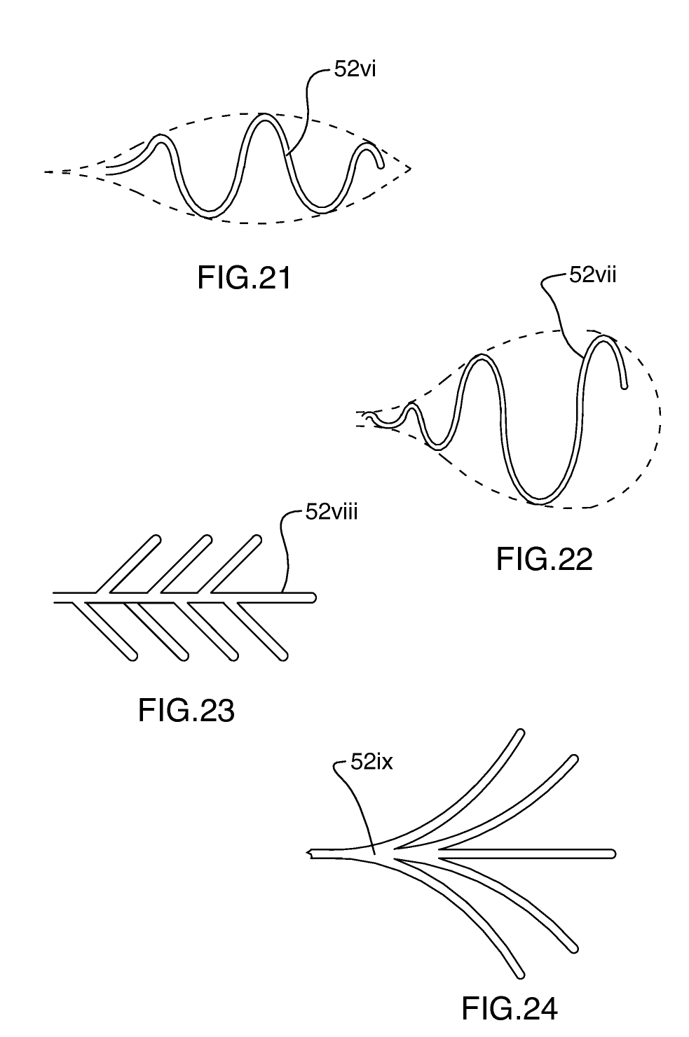

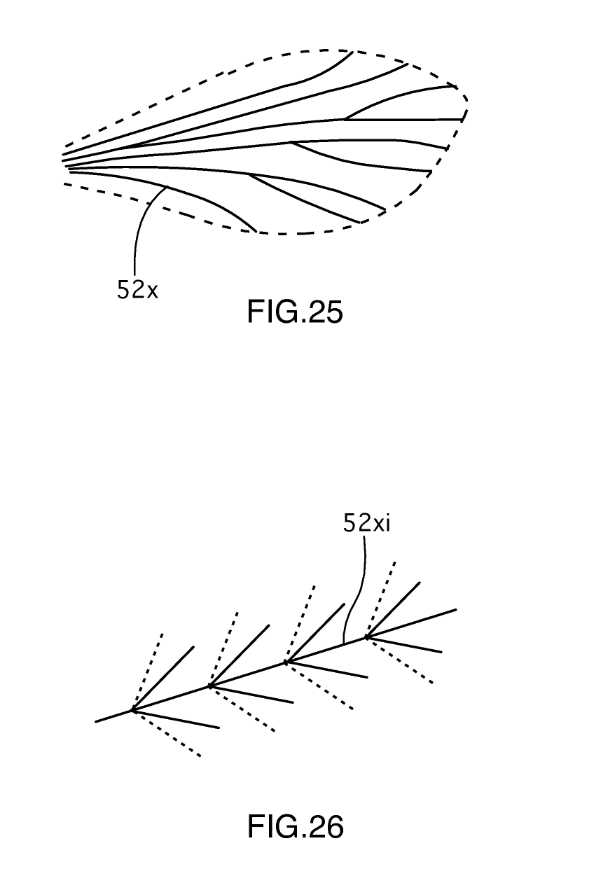

As seen in the drawings, many other configurations of anchoring sections are within the scope of the invention. For example, in some embodiments, the anchoring section is selected from the group consisting of: a substantially disc-shaped anchoring section, for example anchoring section 52i seen in FIG. 16, a substantially teardrop shaped anchoring section, for example anchoring section 52iv seen in FIG. 19, a substantially ellipsoidal anchoring section, for example anchoring section 52iii seen in FIG. 18, a substantially sinusoidal anchoring section, for example anchoring sections 52vi (having a sinusoidal envelope) and 52vii (having a tear-dropped shaped envelope) seen in FIGS. 21 and 22, a substantially pennated anchoring section, for example anchoring section 52viii seen in FIG. 23, a substantially fanned anchoring section, for example anchoring section 52ix seen in FIG. 24, and a substantially reticulated anchoring section, for example anchoring section 52x seen in FIG. 25.

In some embodiments of the invention, the anchoring section is substantially longitudinally deformable, substantially circumferentially deformable, or both substantially longitudinally and substantially longitudinally deformable. This enhances the capability of the stent in which these anchoring sections are formed to expand with body vessel growth. For example, FIG. 26 illustrates a pennated wall supporting member 26xi that is substantially longitudinally deformable.

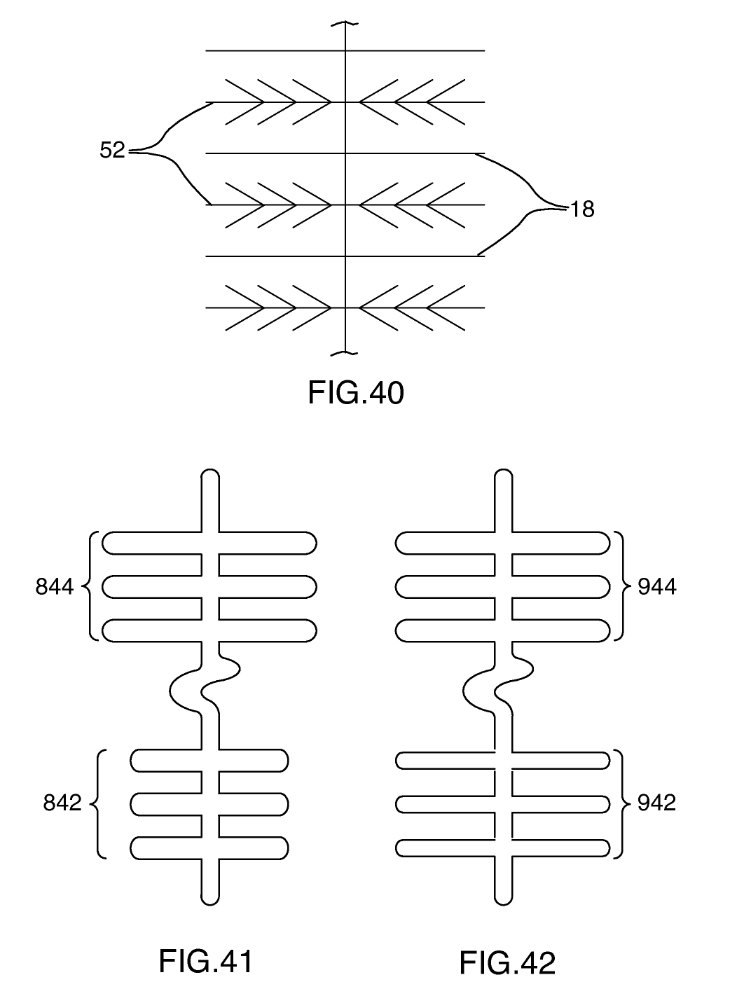

In some embodiments of the invention, only some of the wall supporting members include an anchoring section. For example, as seen schematically in FIG. 40, wall supporting members alternate longitudinally between wall supporting members 52 including a deformable anchoring section and wall supporting member 18 that do not include an anchoring section. The wall supporting members 18 may then be substantially rectilinear or substantially tapered towards their free ends, for example. This configuration reduces the minimal distance required between adjacent wall supporting members so that the wall supporting members that include a deformable anchoring section may deform while preserving a sufficient stent radial rigidity.

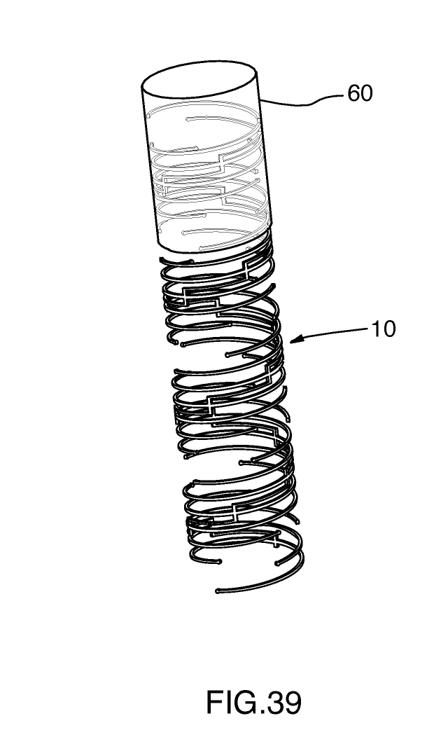

In some embodiments of the invention, the stent 10 is made out of shape-memory alloys so that the stent 10 is self-expandable when inserted into the body vessel. Alternatively, the stent 10 is balloon-expandable and must therefore be expanded through the use of a balloon catheter. In some embodiments of the invention, as seen in FIG. 39, a sheath 60 covers the stent 10. For example, the sheath 60 is mechanically coupled to the stent 10. Also, in some embodiments of the invention, the stent 10 is made out of a biodegradable material, such as for example poly-l-lactic acid (PLLA), poly-lactic-co-glycolic acid (PGLA), magnesium or a magnesium alloy. Also, in yet other alternative embodiments of the invention, the stent 10 is a drug eluting stent.

In use, the stent 10 is inserted into a body vessel. Then, the stent 10 is expanded from a stent retracted configuration to a stent expanded configuration. To that effect, the wall supporting members 18 are expanded from their respective retracted configurations to their respective expanded configurations.

It is hypothesized that afterwards, the natural healing process of the vessel wall embeds the stent 10 into the vessel wall. When the body vessel grows, the stent 10 expands with the body vessel because of the relatively low resilience provided by the shape of the wall supporting members 18, which allows the wall supporting members to remain embedded within the vessel wall and be carried during growth.

FIG. 9 illustrates an alternative embodiment of the invention wherein a stent 810 includes wall supporting members 818 that extend from an alternative backbone 812. The backbone 812 is substantially elongated, but takes the form of a substantially helicoidal member that is wound about the stent longitudinal axis of the stent 810. This shape of the backbone 812 typically increases the flexibility of the stent 810 so that the stent 810 is relatively easily movable through the body and other vessels through which the body vessel is accessed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

The embodiments of the invention described above are intended to be exemplary only. The scope of the invention is therefore intended to be limited solely by the scope of the appended claims.

What is claimed is:

1. A stent insertable in a body vessel, said body vessel defining a vessel wall, said stent comprising:
a plurality of struts, said struts defining a substantially elongated stent passageway, said struts being configured, sized and operatively coupled to each other in a manner such that said stent is deformable-longitudinally and radially between a first configuration and a second configuration;
wherein in said first configuration, said stent passageway has a first radial dimension and a first longitudinal dimension and in said second configuration, said stent has a second radial dimension and a second longitudinal dimension;
said second radial dimension being larger than said first radial dimension and said second longitudinal dimension being larger than said first longitudinal dimension;
wherein, after having been inserted in said body vessel, said stent is able to expand substantially longitudinally and radially with said body vessel as said body vessel grows.

2. A stent as defined in claim 1, wherein said struts define a circumferentially interrupted structure.

3. A stent as defined in claim 1, wherein said struts define
a generally longitudinally extending backbone, said backbone being deformable between a backbone shorter configuration and a backbone longer configuration, wherein said backbone extends longitudinally along a longer distance in said backbone longer configuration than in said backbone shorter configuration; and
a wall supporting member for supporting said vessel wall, said wall supporting member extending substantially circumferentially from said backbone, said wall supporting member being circumferentially interrupted and defining a free end, said wall supporting member being substantially radially expandable between a supporting member retracted configuration and a supporting member expanded configuration, wherein upon an expansion of said wall supporting member, said free end moves relatively to said backbone.

4. A stent as defined in claim 3, wherein said wall supporting member defines an anchoring section for anchoring said wall supporting member to said vessel wall.

5. A stent as defined in claim 4, wherein said anchoring section is located substantially adjacent said free end.

6. A stent as defined in claim 3, wherein said wall supporting member includes a fixed end located substantially adjacent said backbone, said wall supporting member having a cross sectional configuration that varies between said fixed and free ends.

7. A stent as defined in claim 6, wherein said wall supporting member is tapered in a direction leading from said free end towards said fixed end wherein the cross-sectional area of said wall supporting member increases with increasing distance from said fixed end.

8. A stent as defined in claim 3, comprising:
at least two wall supporting members for supporting said vessel wall, each of said at least two wall supporting members extending substantially circumferentially from said backbone, each of said at least two wall supporting members being
circumferentially interrupted and defining a respective free end; and
substantially radially expandable between a respective retracted configuration and a respective expanded configuration;
wherein two wall supporting members from said at least two wall supporting members extend in substantially opposite directions from said backbone.

9. A stent as defined in claim 3, wherein said backbone includes a thinned out section defining a substantially deformable region.

10. A stent as defined in claim 3, said backbone including a first substantially longitudinally extending section, a second substantially longitudinally extending section substantially spaced apart from said first substantially longitudinally extending section and a thinned out section extending therebetween, said thinned out section having a cross-sectional area that is substantially smaller than a cross-sectional area of the first and second longitudinally extending sections, said thinned out section thereby defining a substantially deformable section.

11. A stent as defined in claim 10, wherein said first and second substantially longitudinally extending sections are substantially circumferentially spaced apart from each other by an angle of about 45 degrees to about 180 degrees.

12. A stent as defined in claim 10, wherein said backbone includes a linking segment extending between said first and second substantially longitudinally extending sections, said linking segment being substantially arc segment shaped.

13. A stent as defined in claim 10, further comprising a first wall supporting member and a second wall supporting member, said first wall supporting member extending substantially circumferentially from said first longitudinally extending section and said second wall supporting member extending substantially circumferentially from said second longitudinally extending section.

14. A stent as defined in claim 3, comprising a plurality of wall supporting members for supporting said vessel wall, said wall supporting members being substantially longitudinally spaced apart from each other, each of said wall supporting members extending substantially circumferentially from said backbone, each of said wall supporting members being circumferentially interrupted and defining a respective free end, each of said wall supporting members being substantially radially expandable between a respective retracted configuration and a respective expanded configuration.

15. A stent as defined in claim 14, wherein said wall supporting members form a radially most rigid region and a radially least rigid region, said radially least and most rigid regions being substantially longitudinally spaced apart from each other, said wall supporting members being longitudinally spaced apart by a smaller distance within said radially least rigid region than within said radially most rigid region.

16. A stent as defined in claim 14, wherein
said stent defines a stent first end, a substantially longitudinally opposed stent second end and a stent midpoint located therebetween; and
a substantially longitudinal distance between adjacent wall supporting members from said plurality of wall supporting members varies from at least one of said stent first and second ends towards said stent midpoint.

17. A stent as defined in claim 3, further comprising a sheath mechanically coupled to at least some of said wall supporting members.

18. A stent as defined in claim 1, wherein said second longitudinal dimension is at least about 2.5 times larger than said first longitudinal dimension and said second radial dimension is at least about 2.5 times larger than said first radial dimension.

19. A stent as defined in claim 11, where said angle is selected from the group consisting of: about 45 degrees, about 60 degrees, about 120 degrees and about 180 degrees.
